(19) United States
(12) Patent Application Publication
(10) Pub. No.: US 2026/0318904 A1
(43) Pub. Date: Oct. 15, 2026

(54) EXAMINATION DEVICE

(71) Applicant: FUJIFILM CORPORATION, Tokyo (JP)

(72) Inventor: Shunsuke NISHIMURA, Tokyo (JP)

(21) Appl. No.: 19/464,392

(22) Filed: Jan. 29, 2026

(30) Foreign Application Priority Data
Feb. 12, 2025 (JP) 2025-021100

(51) Int. Cl. G01N 33/53; G01N 21/76

(52) U.S. Cl. CPC G01N 33/5302; G01N 21/76

(57) ABSTRACT

The examination device that examines a specimen includes an inclined surface that is connected to an upper surface of a housing and is inclined rearward at a front surface of the housing, in which, in a case in which an angle with respect to a horizontal direction is denoted by α, a condition of 90°<α<160° is satisfied, and a door that is openable and closable and is disposed below the inclined surface at the front surface, in which an opening operation from a closed position toward an open position starts with an operation of moving downward along the inclined surface.

### CROSS-REFERENCE TO RELATED APPLICATIONS

The present application claims priority under 35 U.S.C. §119 to Japanese Patent Application No. 2025-021100, filed on Feb. 12, 2025. The above application is hereby expressly incorporated by reference, in its entirety, into the present application.

### BACKGROUND

### 1. Technical Field

The technology of the present disclosure relates to an examination device.

### 2. Description of the Related Art

An examination device that examines a specimen such as blood is known. Such an examination device is also called an analysis device, a measurement device, or the like. A door that is openable and closable is provided in a housing of the examination device. In the examination device, in a case in which the door is opened, a working space can be accessed, and the door is often provided on a front surface of the housing (see JP2019-178867A and the like).

In the examination device described in JP2019-178867A, about half of an upper portion of the front surface of the housing is a door, and the door slides upward to be opened. In addition, in the examination device described in JP2019-178867A, a display section that displays information related to the examination, an operation section, and the like are arranged on the front surface of the housing.

A stage on which the specimen is set is provided in the working space. In a case in which a user such as an operator who performs the examination performs work related to the examination, the user stands on a front surface side of the housing, opens the door at the front surface, sets the specimen, and performs an operation such as setting the specimen and a reagent to execute the examination.

### SUMMARY

The technology of the present disclosure provides an examination device having improved usability compared to the related art.

A first technology of the present disclosure relates to an examination device that examines a specimen, the examination device including: an inclined surface that is connected to an upper surface of a housing and is inclined rearward on a front surface of the housing, in which, in a case in which an angle with respect to a horizontal direction is denoted by α, a condition of 90°<α<160° is satisfied; and a door that is openable and closable and is disposed below the inclined surface at the front surface, in which an opening operation from a closed position toward an open position starts with an operation of moving downward along the inclined surface.

A second technology of the present disclosure relates to an examination device that examines a specimen, in which a display section and a door that is openable and closable are arranged in this order from above on a front surface of a housing, a working space having a stage on which the specimen is set is provided inside the door, and an opening operation of the door from a closed position toward an open position starts with an operation of moving obliquely downward and forward and the door descends toward the open position.

A third technology of the present disclosure relates to an examination device that examines a specimen, in which a door that is openable and closable is disposed on a front surface of a housing, a working space having a stage on which the specimen is set is provided inside the door, and in an open position of the door, an upper end surface of the door and an upper surface of the stage are positioned on the same plane.

The technology of the present disclosure can improve usability compared to the related art.

### BRIEF DESCRIPTION OF THE DRAWINGS

FIG. 1 is a perspective view showing an appearance of an examination device in a state in which a door is in a closed position.

FIG. 2 is a perspective view showing an appearance of the examination device in a state in which the door is in an open position.

FIG. 3 is a side view of the examination device in a state in which the door is in the closed position.

FIG. 4 is a side view of the examination device in a state in which the door is open.

FIG. 5 is a perspective view showing a working space.

FIG. 6 is a view showing a door including a pair of arms.

FIG. 7 is a view showing a door in a state in which the door is in the closed position.

FIG. 8 is a view showing a state of the door immediately after the opening operation is started.

FIG. 9 is a view showing a state in which the door is further opened from the state of FIG. 8.

FIG. 10 is a view showing a state in which the door is further opened from the state of FIG. 9 and protrudes most forward.

FIG. 11 is a view showing a door in a state in which the door is in the open position.

### DETAILED DESCRIPTION

### First Embodiment

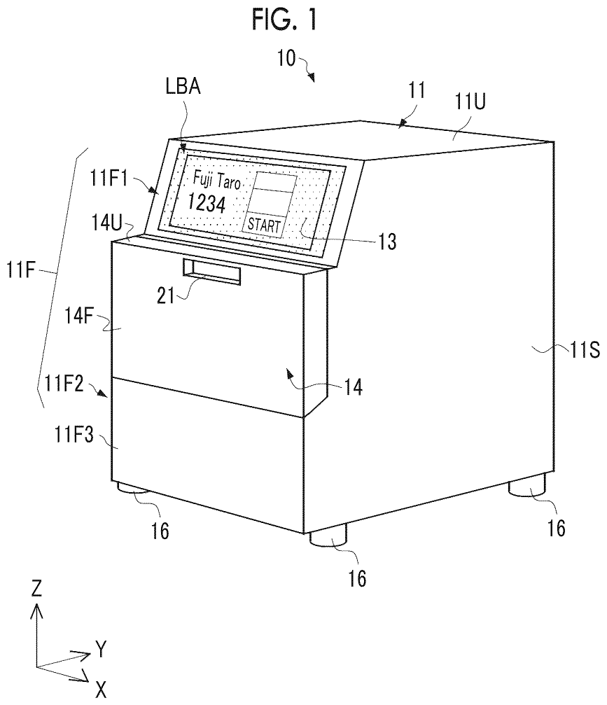

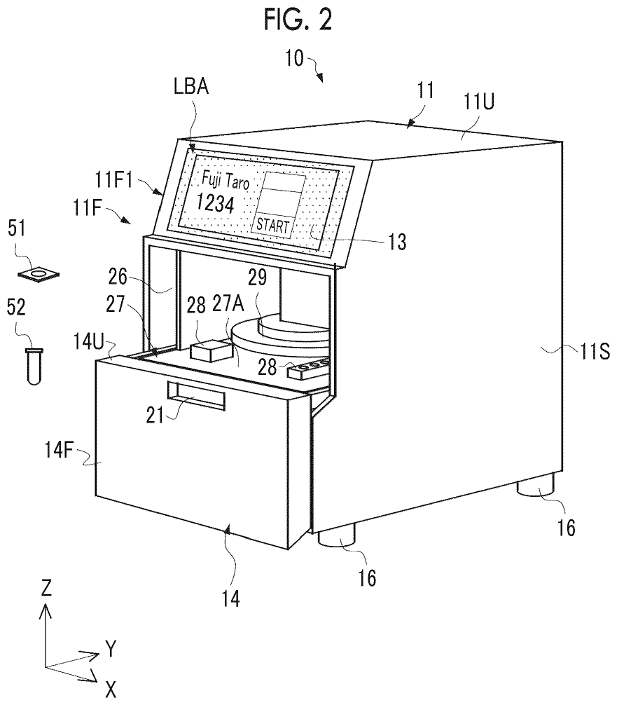

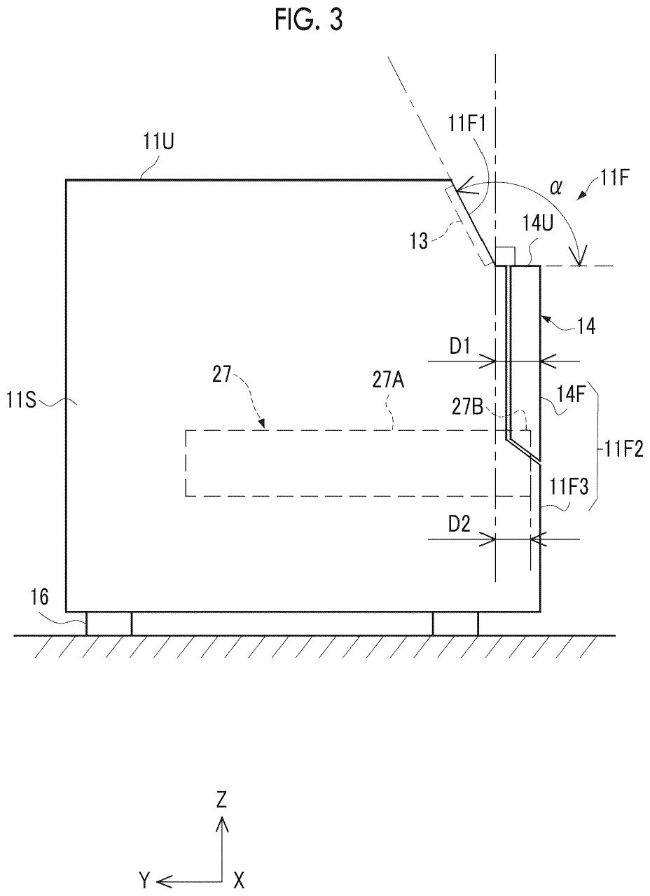

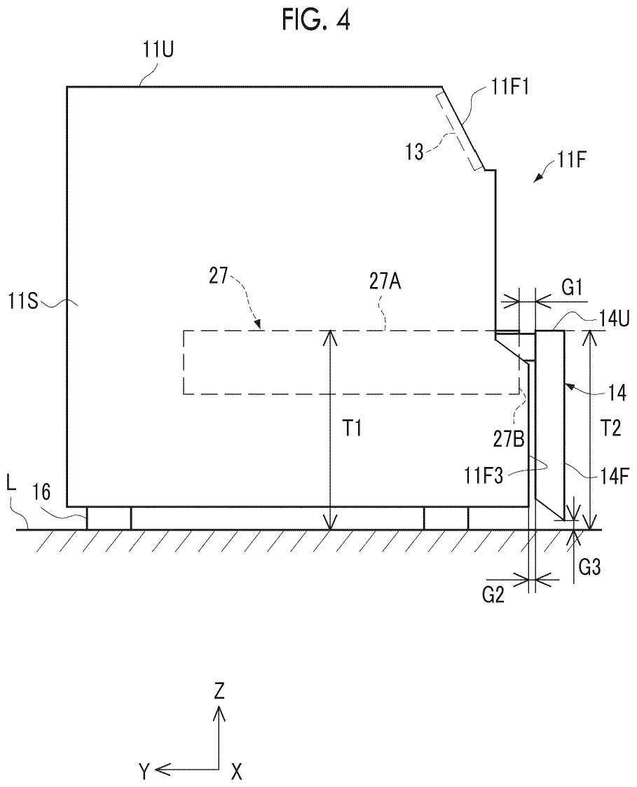

FIGS. 1 and 2 are perspective views showing an appearance of an examination device 10 according to an embodiment of the present disclosure. FIG. 1 is a view in a state in which a door 14 is closed, and FIG. 2 is a view in a state in which the door 14 is open. FIG. 3 is a side view of the examination device 10 corresponding to FIG. 1 in a state in which the door 14 is closed. FIG. 4 is a side view of the examination device 10 corresponding to FIG. 2 in a state in which the door 14 is open.

The examination device 10 is an examination device that examines a specimen collected from a living body such as a human or an animal. The examination device 10, for example, spots the specimen on an analysis chip on which a reagent is immobilized, and detects an examination target substance by using an antigen-antibody reaction between the examination target substance in the specimen and the reagent. As an examination method, for example, various methods such as a method of measuring a reaction state between a dry reagent and the specimen by colorimetric analysis and a method of measuring an antigen-antibody reaction by surface plasmon fluorescence measurement are available. The specimen is, for example, a body fluid such as blood collected from the living body. The examination target substance included in the specimen is an antigen, an antibody, a protein, a low-molecular-weight compound, and the like. An analysis chip 51 shown in FIG. 2 is an example of an analysis chip on which a dry reagent is immobilized, and a container 52 shown in FIG. 2 is an example of a container that accommodates the specimen.

The examination device 10 is, for example, an examination device having a relatively small housing installed in a veterinary hospital or the like, and is used to examine a specimen collected from an animal. The examination device 10 can be applied to various in vitro examination devices that examine the specimen in vitro, and may be applied to, for example, in addition to the above, an immunological analysis device based on a basic principle of a chemiluminescence enzyme immunoassay method. The technology of the present disclosure is not limited to the use and the examination method as long as the examination device examines the specimen.

A housing 11 of the examination device 10 has, for example, a substantially rectangular parallelepiped shape as a whole. The housing 11 has an outer peripheral surface formed by a plurality of surfaces including a front surface 11F, a rear surface (not shown), an upper surface 11U, a lower surface (not shown), and two side surfaces 11S on the left and right. In coordinates shown in FIGS. 1 and 2, an X direction is a width direction of the housing 11. In addition, a Y direction is a front-rear direction or a depth direction of the housing 11, and a Z direction is an up-down direction or a height direction of the housing 11.

The front surface 11F is connected to the upper surface 11U at an upper portion. The two side surfaces 11S connect the front surface 11F and the upper surface 11U in each of the width directions of the front surface 11F and the upper surface 11U. The upper surface 11U and the two side surfaces 11S are planes. The upper surface 11U is composed of a plane extending horizontally, and the two side surfaces 11S are composed of planes extending vertically. A connector for connecting a cable or the like and an exhaust port for heat dissipation are disposed on the rear surface. Leg portions 16 and the like are provided on the lower surface.

The front surface 11F has an inclined surface 11F1 and a vertical plane 11F2. The inclined surface 11F1 is an inclined surface that is inclined rearward. The “inclined rearward” refers to being inclined such that the upper end is positioned rearward of the lower end. In other words, the inclined surface 11F1 has a shape as if it were chamfered by cutting a corner portion present at a boundary between the front surface 11F and the upper surface 11U of the housing 11.

As shown in FIG. 3, in a case in which an angle with respect to a horizontal direction (the Y direction in FIG. 3) is denoted by α, the inclined surface 11F1 satisfies a condition of 90°<α<160°. Since a vertical direction (the Z direction in FIG. 3) is 90°, the “α being greater than 90°” means that the inclined surface 11F1 is inclined rearward. In addition, the angle α more preferably satisfies a condition of 90°<α<145°, and still more preferably satisfies a condition of 100°<α<120°.

In addition, a display section 13 that displays information related to the examination is provided on the inclined surface 11F1. The information related to the examination includes identification information for identifying the specimen, a progress status of the examination, an examination result, and the like. In addition, the display section 13 is, for example, a touch panel display, and displays operation buttons and the like and is also used as an operation section. As the display section 13, for example, a liquid crystal display is used.

The inclined surface 11F1 has a low brightness region LBA (hatched in the drawing) that includes the display section 13 and has a relatively low brightness color. The color of the low brightness region LBA is a color having a lower brightness than the color of the other portions (that is, portions other than the low brightness region LBA) of the front surface 11F, the upper surface 11U, and the two side surfaces 11S of the housing 11. The low brightness region LBA occupies, for example, 80% or more of the area of the inclined surface 11F1, and the color of the low brightness region LBA largely determines the impression of the color of the inclined surface 11F1.

In the present example, the display section 13 has an area smaller than the low brightness region LBA and constitutes a part of the low brightness region LBA. Therefore, the low brightness region LBA is constituted by the display section 13 and a peripheral portion thereof. It should be noted that the entire low brightness region LBA may be constituted by the display section 13.

A background color of the display section 13 is the color of the low brightness region (or a color having the same low brightness as the color of the low brightness region). The background color is a color of a background of information displayed on the display section 13 and a display button or the like. Specifically, the color of the low brightness region LBA is, for example, black. As described above, by setting the background color of the display section 13 to the color of the low brightness region, it is possible to further emphasize information from a light emitting element such as a liquid crystal display (LCD) or a light emitting diode (LED) and improve visibility. Further, in an installation environment (an examination room or the like) of the examination device 10 in which there are many white elements, a high contrast is realized between the installation environment and the display section 13, and it is possible to easily and quickly ascertain a position of the display section 13 by visual observation.

On the other hand, in the front surface 11F, the upper surface 11U, and the two side surfaces 11S, the color of the portion other than the low brightness region LBA is an achromatic color. As is well known, the achromatic color is a color that is not expressed by a hue or a chroma saturation and is expressed only by brightness, and is white, black, gray, and the like. In the present example, the color of the portion other than the low brightness region LBA is white, and a tone of the housing 11 is monochromatic by a combination of the white and the black that is the color of the low brightness region LBA. As described above, the color of the low brightness region LBA and the color of the portion other than the low brightness region LBA are colored to have a high contrast. In addition, in order to give a clear impression, the coloring of the low brightness region LBA and the other portions is composed of two colors of black and white as an example.

The vertical plane 11F2 is a plane extending vertically. In addition, as shown in FIG. 3, a plane extending horizontally (a surface including an upper end surface 14U) is provided between a lower end of the inclined surface 11F1 and an upper end of the vertical plane 11F2. Therefore, as indicated by a distance D1 in FIG. 3, the inclined surface 11F1 is positioned rearward of the vertical plane 11F2.

A door 14 that is openable and closable is provided on the front surface 11F, and a front surface portion 14F of the door 14 constitutes a part of the vertical plane 11F2. The door 14 is formed over an entire width of the housing 11. Then, the upper end surface 14U of the door 14 is exposed and constitutes a part of a plane extending horizontally that connects the lower end of the inclined surface 11F1 and the upper end of the vertical plane 11F2. That is, the upper end surface 14U of the door 14 is closer to horizontal than the inclined surface 11F1, and is a horizontal surface in the present example.

The door 14 is openable and closable between the closed position shown in FIG. 1 and the open position shown in FIG. 2. Although an opening and closing mechanism and detailed operations of the door 14 will be described later, the door 14 descends from the closed position to the open position in the opening operation.

In addition, in the closed position, a plane including the upper end surface 14U of the door 14 is adjacent to the lower end of the inclined surface 11F1. Here, the “adjacent” refers to a state in which the two surfaces are adjacent to each other without an interposed object. Of course, a gap may be present.

In addition, the front surface portion 14F of the door 14 that is connected to the upper end surface 14U and extends downward is closer to vertical than the inclined surface 11F1. In the present example, since the front surface portion 14F constitutes a part of the vertical plane 11F2, the front surface portion 14F is a vertical surface.

In addition, the front surface 11F of the housing 11 has a lower front surface 11F3 adjacent to a lower side of the door 14 in the closed position. The lower front surface 11F3 also constitutes the vertical plane 11F2 as in the front surface portion 14F, and as shown in FIG. 3, the front surface portion 14F and the lower front surface 11F3 are positioned on the same plane in the closed position of the door 14. A gap is present between the front surface portion 14F and the lower front surface 11F3, but there is no step. Here, the “same plane” is a concept including, for example, an allowable error of about plus or minus 5 mm in addition to being completely the same.

In addition, in the door 14, a finger hook 21 for opening and closing the door 14 is provided on the front surface portion 14F on an upper side of a center in an up-down direction. The finger hook 21 is formed of a recessed portion, for example, but may be formed of a protruding portion.

As shown in FIG. 2, a working space 26 on which the specimen is set is provided inside the door 14. A stage 27 on which the specimen is set is provided at a lower end of the working space 26. A set portion 28 on which the analysis chip 51, the container 52 of the specimen, and the like are set, an incubator 29 that accommodates the analysis chip 51 on which the specimen is spotted after the specimen is spotted on the analysis chip 51, and the like are provided on an upper surface 27A of the stage 27. In addition, a suction and discharge mechanism (not shown) that has a nozzle that sucks the specimen from the container 52 and spots the sucked specimen on the analysis chip 51 is provided in the working space 26.

As described above, the display section 13 and the door 14 that is openable and closable are arranged in this order from above on the front surface portion 14F of the housing 11, and the working space 26 having the stage 27 on which the specimen is set is provided inside the door 14.

As shown in FIG. 3, in the depth direction (the Y direction in FIG. 3) of the housing 11, a front end 27B of the stage 27 protrudes forward of the display section 13. In addition, as indicated by a distance D2 in FIG. 3, the front end 27B of the stage 27 protrudes forward of the lower end of the inclined surface 11F1. The lower end of the inclined surface 11F1 is an intersection between the inclined surface 11F1 and an extension line of the upper end surface 14U of the door 14 in FIG. 3. In addition, in the closed position of the door 14, the front surface portion 14F of the door 14 is positioned forward of the display section 13.

In addition, as shown in FIG. 4 (see also FIG. 2), in the open position of the door 14, the upper end surface 14U of the door 14 and the upper surface 27A of the stage 27 are in a state of being arranged in the depth direction (that is, the Y direction) of the housing 11. More specifically, the upper end surface 14U of the door 14 and the upper surface 27A of the stage 27 are positioned on the same plane. That is, a height T1 from an installation surface L of the housing 11 to the upper surface 27A of the stage 27 and a height T2 from the installation surface L to the upper end surface 14U of the door 14 are the same. Here, the “same plane” is a concept including, for example, an allowable error of about plus or minus 5 mm in addition to being completely the same.

In addition, the upper end surface 14U of the door 14 and the front end 27B of the stage 27 are disposed close to each other. Specifically, in the open position of the door 14, a gap G1 between the upper end surface 14U and the front end 27B of the stage 27 is 10 mm or less. Statistically, a minimum thickness of a third joint of a fingertip of an adult is 10 mm, and a numerical value of 10 mm or less means a gap to the extent that a finger of a person does not enter from the viewpoint of human engineering.

In the open position, the door 14 covers the lower front surface 11F3 on a front side of the lower front surface 11F3. Then, in the closed position, a gap G2 between the door 14 and the lower front surface 11F3 of the housing 11 is 10 mm or less. The reason for the numerical value of 10 mm or less of the gap G2 is the same as that of the gap G1.

In addition, as shown in FIG. 4, in the open position, the door 14 is restricted in height to stop at a position at which a gap G3 is generated between a lower end of the door 14 and the installation surface L.

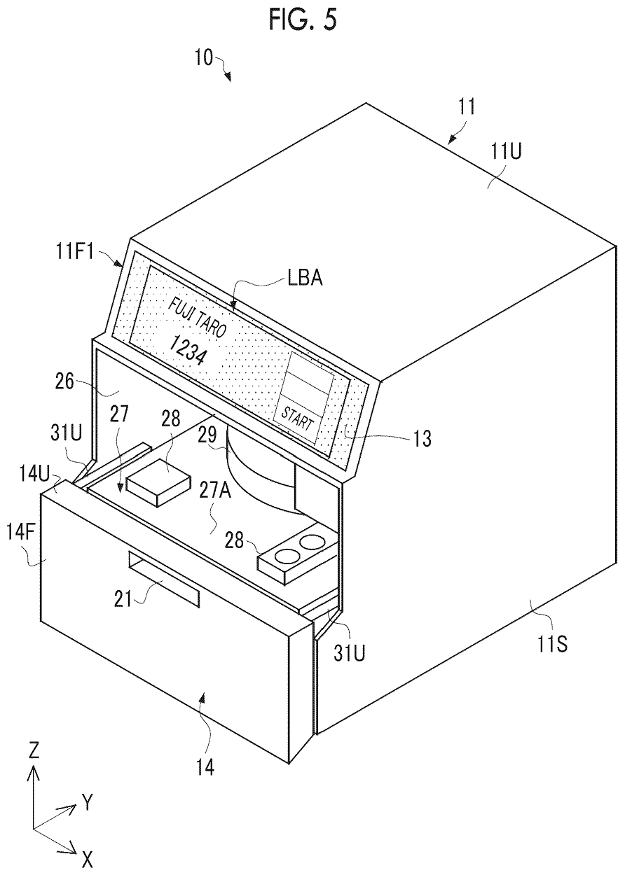

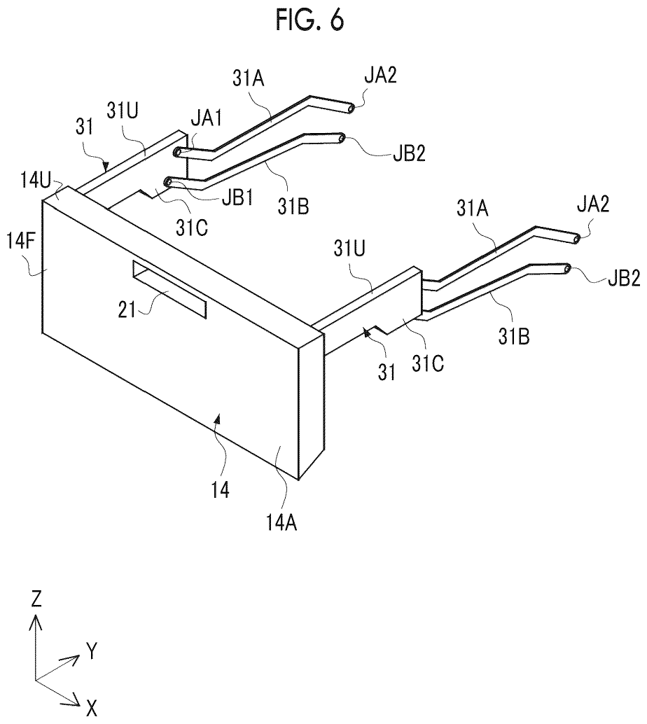

In addition, as shown in FIGS. 5 and 6, the door 14 includes a pair of arms 31 that support the door 14 at both ends in the width direction, and in the open position, upper end surfaces 31U of the pair of arms 31 are in a state of being arranged at both ends of an upper surface 27U of the stage 27 in the width direction (that is, the X direction). That is, as shown in FIG. 5, in the open position, the upper end surface 31U of the arm 31 is at substantially the same height as the upper surface 27U of the stage 27.

As shown in FIG. 6, the pair of arms 31 of the door 14 is provided at both ends of the door 14 in the width direction and extends in the depth direction of the housing 11. The pair of arms 31 constitute the opening and closing mechanism of the door 14 described below. Each of the pair of arms 31 includes a first link 31A, a second link 31B, and a support portion 31C. The support portion 31C is formed of, for example, a plate-shaped member and is disposed such that a thickness direction is along the width direction (that is, the X direction). The support portion 31C is connected to a front end of a body portion 14A having the front surface portion 14F in the door 14. The upper end surface 31U is, for example, an upper end surface of the support portion 31C. A coupling portion JA1 at one end of the first link 31A and a coupling portion JB1 at one end of the second link 31B are rotatably attached to a rear end portion of the support portion 31C.

A coupling portion JA2 is provided at a rear end portion of the first link 31A, and the coupling portion JA2 is rotatably attached to a part of a body of the housing 11. In addition, a coupling portion JB2 is provided at a rear end portion of the second link 31B, and the coupling portion JB2 is rotatably attached to a part of the body of the housing 11.

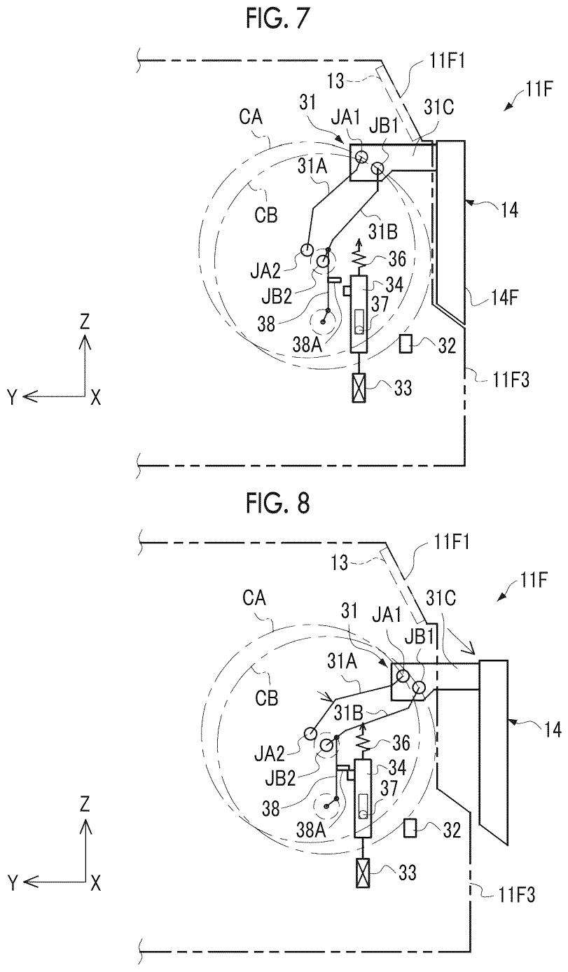

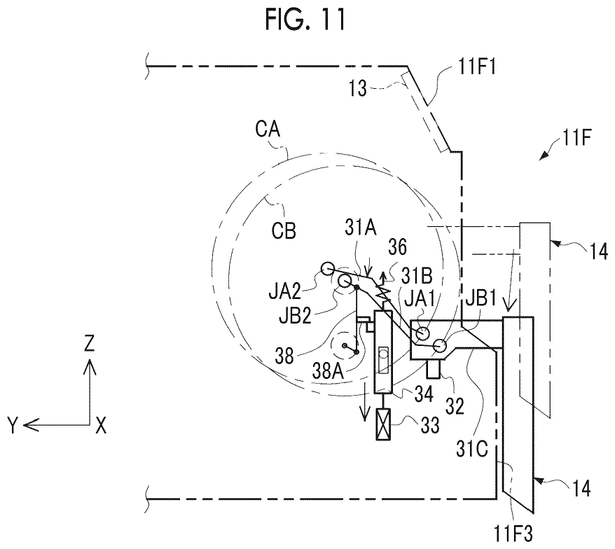

The configuration and the operation of the opening and closing mechanism of the door 14 will be described with reference to FIGS. 7 to 11. FIG. 7 shows a state in which the door 14 is in the closed position, and FIG. 11 shows a state in which the door 14 is in the open position. FIGS. 8 to 10 show a change in the position of the door 14 in the middle of moving from the closed position to the open position. In a case in which the door 14 is opened and closed, the first link 31A rotates about the coupling portion JA2 on the rear end side, and the second link 31B rotates about the coupling portion JB2 on the rear end side. In a case in which the first link 31A rotates, the coupling portion JA1 on the front end side moves along an arc of a part of a circular orbit CA, and in a case in which the second link 31B rotates, the coupling portion JB1 on the front end side moves along an arc of a part of a circular orbit CB. As a result, the door 14 descends from the closed position shown in FIG. 7 to the open position shown in FIG. 11 below the closed position.

By providing the two links of the first link 31A and the second link 31B, the posture of the door 14 is maintained in a constant posture in a case of opening and closing. The door 14 moves to the open position while maintaining the posture of the closed position in which the front surface portion 14F extends in the vertical direction, in the entire movement range in the opening and closing operation.

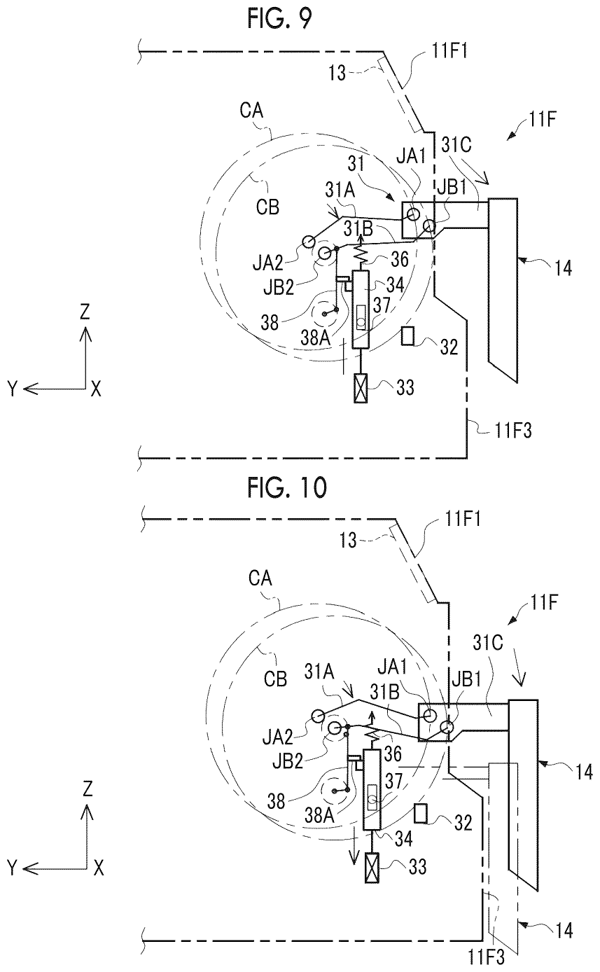

In the closed position, the coupling portions JA1 and JB1 are disposed to be positioned above the center of each of the circular orbits CA and CB, and in the open position, the coupling portions JA1 and JB1 are disposed to be positioned below the center of each of the circular orbits CA and CB. Then, the movement range of the coupling portions JA1 and JB1 from the closed position to the open position is less than a half of the circular orbits CA and CB on the front end side and is less than 180° in terms of a central angle of the circular orbits CA and CB. That is, the coupling portions JA1 and JB1 are positioned at a highest point in the height direction (that is, the Z direction) in the closed position and are positioned at a lowest point in the open position. Then, the opening operation of the door 14 from the closed position toward the open position starts with an operation of moving obliquely downward and forward along the tangent direction of the circular orbits CA and CB as shown in FIGS. 8 and 9. The “obliquely downward and forward” refers to a direction in which the movement is made to the front surface portion 14F side of the housing 11 while descending. Thereafter, at an approximately middle point between the closed position and the open position shown in FIG. 10, the coupling portions JA1 and JB1 are positioned at the most front end side. The orientation of the tangent of the circular orbits CA and CB at this time is in the vertical direction. After passing the middle point shown in FIG. 10, as shown in FIG. 11, the orientation of the tangent of the circular orbits CA and CB is inclined rearward, and the coupling portions JA1 and JB1 descend while moving rearward.

The door 14 is opened and closed by the rotational movement of the first link 31A and the second link 31B. Then, the movement trajectory of the door 14 from the closed position toward the open position is determined by setting the length of the first link 31A and the second link 31B that determine the radii of the circular orbits CA and CB, the position on the circular orbits CA and CB in the open position and the closed position of the coupling portions JA1 and JB1, and the like. The opening operation of the door 14 from the closed position toward the open position starts with an operation of moving downward along the inclined surface 11F1. Here, the “along the inclined surface 11F1” is not limited to a direction that completely coincides with the inclination direction of the inclined surface 11F1, and includes a range that can be said to be obliquely downward and forward, which is an approximate direction of the inclination direction of the inclined surface 11F1. That is, as shown in FIGS. 8 and 9, the operation of the door 14 obliquely downward and forward is also included in the operation along the inclined surface 11F1.

In addition, in the entire movement range in the opening and closing operation of the door 14, a rear end of the door 14 does not protrude forward of the front end of the door 14 in a case in which the door 14 is in the open position. Here, the front end and the rear end of the door 14 are the front end and the rear end in the depth direction (that is, the Y direction) of the housing 11. The door 14 protrudes most forward of the housing 11 on the front side at the position shown in FIG. 10 in the movement range of the opening and closing operation. Even at this position, the rear end of the door 14 is positioned rearward of the position of the front end of the door 14 (that is, the front surface portion 14F) in the open position.

The movement trajectory of the door 14 is as described above, but in the opening and closing mechanism of the door 14, various improvements have been made to improve the operation feeling of the door 14 and to ensure smooth operation. First, as described above, in the open position of the door 14, a gap G3 is ensured between the door 14 and the installation surface L, but a stopper 32 for ensuring the gap G3 is provided in the opening and closing mechanism. For example, the door 14 is stopped by the support portion 31C of the arm 31 abutting on the stopper 32 in the open position. In addition, the door 14 is configured to engage with the body of the housing 11 at the closed position by an engagement portion (not shown). As a result, the door 14 is prevented from being unintentionally opened.

In addition, the door 14 descends in the opening operation. In the opening and closing mechanism, in order to suppress the door 14 from rapidly descending due to the action of gravity, a damper 33 for attenuating a force in a downward direction due to the self-weight of the door 14 is provided. An interlocking member 34 is connected to the damper 33. The interlocking member 34 is a member for operating the damper 33 in conjunction with the opening operation of the door 14. A long hole extending in the up-down direction is provided in the interlocking member 34, and the long hole is engaged with a pin 37. The pin 37 is fixed to the body of the housing 11. The interlocking member 34 is movable in the up-down direction, but the movement range is limited by the engagement between the pin 37 and the long hole. Then, the lower end of the interlocking member 34 is connected to the damper 33, while the upper end of the interlocking member 34 is biased upward by a tension spring 36.

On the other hand, a sub-link 38 having a lever 38A that operates the damper 33 is connected to the middle of the second link 31B. One end of the sub-link 38 is rotatably attached to the second link 31B, and the other end of the sub-link 38 is rotatably fixed to the body of the housing 11. The sub-link 38 has a rotatable joint at an intermediate position. In a case in which the second link 31B rotates, the sub-link 38 also rotates about the other end. In the closed position shown in FIG. 7, the lever 38A is spaced from the interlocking member 34, but in a case in which the second link 31B rotates to the position shown in FIG. 8, the lever 38A abuts on the interlocking member 34 to move the interlocking member 34 downward. By the action of the lever 38A, the interlocking member 34 descends against the tensile force of the tension spring 36. In a case in which the interlocking member 34 descends, the damper 33 is compressed to attenuate the force of the interlocking member 34 in the downward direction. By such an action, the door 14 moves toward the open position with a relatively light force at the initial stage of the start of the opening operation, but in a case in which the lever 38A exceeds the position shown in FIG. 8 at which the lever 38A abuts on the interlocking member 34, a resistance force is applied by the restoring force of the tension spring 36 and the damping force of the damper 33.

As a result, the door 14 is suppressed from rapidly descending to the open position shown in FIG. 11. In addition, by applying an appropriate resistance in the opening operation of the door 14, a favorable operation feeling is provided to the user. In addition, in a case in which the door 14 is closed, in a process of moving from the open position to the closed position, a force in an extension direction of the damper 33 and a contraction force of the tension spring 36 act as an assist force in a case in which the door 14 is pulled upward, which contributes to reducing the operation force of the user. Further, a coupling member that couples the pair of arms 31 on the left and right of the door 14 is provided, and the twisting of the pair of arms 31 in the opening and closing operation is suppressed. As a result, the door 14 can be smoothly operated.

Hereinafter, the action of the above-described configuration will be described. In a case in which the examination is executed, the user stands on the front surface 11F side of the housing 11 and inputs the information related to the examination by operating the display section 13 that functions as the touch panel display. Thereafter, for example, the user performs an operation of opening the door 14 by pressing the upper end surface 14U of the door 14 to move the door 14 in a direction of being pushed down. In a case in which the door 14 is opened, the working space 26 appears. The user sets the analysis chip 51, the container 52 of the specimen, and the like on the stage 27 in the working space 26. Then, for example, the door 14 is closed by hooking a finger on the finger hook 21 of the door 14 to pull the door 14 upward. Thereafter, the execution of the examination is instructed by operating the display section 13. The progress status of the examination and the like are displayed on the display section 13. In a case in which it is confirmed that the examination is completed by the display of the display section 13, the user opens the door 14 again and collects the container 52 of the specimen and the used analysis chip 51 from the stage 27.

Hereinafter, the effects of the first to third technologies of the present disclosure included in the above-described embodiment will be described.

### Effects of First Technique

In the first technology of the present disclosure, as shown in FIGS. 1 to 4 and FIGS. 7 to 11, the examination device 10 includes the inclined surface 11F1 and the door 14 that is openable and closable on the front surface 11F of the housing 11. The inclined surface 11F1 is connected to the upper surface 11U of the housing 11 and is inclined rearward, and in a case in which an angle with respect to the horizontal direction is denoted by α, satisfies a condition of 90°<α<160°. In addition, the door 14 is disposed below the inclined surface 11F1, and the opening operation from the closed position toward the open position starts with the operation of moving downward along the inclined surface 11F1.

Therefore, the user can intuitively understand the operation direction of the door 14, and the usability is improved as compared with the related art that does not have such a configuration. In addition, since the door 14 moves along the inclined surface 11F1, a visual sense of unity is realized between the inclined surface 11F1 and the movement direction of the door 14, and a psychologically favorable operation feeling is provided.

It should be noted that the inclined surface 11F1 is more preferably an inclined surface that satisfies a condition of 90°<α<145°, and still more preferably an inclined surface that satisfies a condition of 100°<α<120°. This is because, for example, in a case in which the display section 13 is provided on the inclined surface 11F1, it is considered that the range of α is preferably at this level in consideration of the visibility of the user.

In addition, in the first technology, as shown in FIGS. 1 to 4 as an example, in the closed position, the upper end surface 14U of the door 14 is exposed, the upper end surface 14U is closer to horizontal than the inclined surface 11F1, and more specifically, the upper end surface 14U is a horizontal surface. Therefore, the door 14 can be opened by pressing the upper end surface 14U of the door 14 downward. Since the upper end surface 14U is closer to horizontal than the inclined surface 11F1, the upper end surface 14U is easily pressed downward. Since the door 14 can be opened by the operation of pressing the upper end surface 14U of the door 14, the operability is also favorable. It should be noted that the upper end surface 14U is preferably the horizontal surface, but may not be the horizontal surface, and the favorable operability can be ensured as long as the upper end surface 14U is closer to horizontal than the inclined surface 11F1.

In addition, in the first technology, as shown in FIGS. 1 to 4 as an example, in the closed position, a plane including the upper end surface 14U of the door 14 is adjacent to the lower end of the inclined surface 11F1. Therefore, it is possible to more conspicuously provide a visual sense of unity between the inclined surface 11F1 and the movement direction of the door 14. In addition, since the inclined surface 11F1 is a characteristic form in the housing 11, the line of sight is likely to be directed toward the inclined surface 11F1. Therefore, the door 14 is included in the visual field by only moving the line of sight below the inclined surface 11F1, so that it is easy to ascertain the position of the door 14.

In addition, in the first technology, as shown in FIGS. 1 to 4 as an example, the front surface portion 14F of the door 14 that is connected to the upper end surface 14U and extends downward is closer to vertical than the inclined surface 11F1, and more specifically, is a vertical surface. Therefore, since the upper end surface 14U close to horizontal is present between the front surface portion 14F of the vertical surface and the inclined surface 11F1, the presence of the upper end surface 14U is visually conspicuous. As described above, the upper end surface 14U is a part that is used for the operation of pressing the door 14 in a case of opening the door 14. In a case in which the upper end surface 14U is conspicuous, the user is easily guided to the upper end surface 14U, and favorable operability is obtained.

In addition, in the first technology, as shown in FIG. 3 as an example, the front surface 11F of the housing 11 has the lower front surface 11F3 adjacent to the lower side of the door 14 in the closed position, and in the closed position, the front surface portion 14F and the lower front surface 11F3 are positioned on the same plane. Therefore, in the closed position, the front surface portion 14F of the door 14 and the lower front surface 11F3 of the housing 11 are flat as a whole, so that the front surface 11F of the housing 11 can be designed to be clear.

In addition, in the first technology, as shown in FIGS. 7 to 11 as an example, in the entire movement range in the opening and closing operation of the door 14, the rear end of the door 14 does not protrude forward of the front end of the door 14 in a case in which the door 14 is in the open position. Therefore, the space in front of the housing 11 required for opening and closing the door 14 can be reduced, and the space for installing the housing 11 can be reduced and the space in front of the housing 11 can be effectively used.

In addition, in the first technology, as shown in FIG. 4 as an example, the front surface 11F of the housing 11 has the lower front surface 11F3 adjacent to the lower side of the door 14 in the closed position, and in the open position, the door 14 covers the lower front surface 11F3 on a front side of the lower front surface 11F3, and a gap G2 between the door 14 and the lower front surface 11F3 of the housing 11 is 10 mm or less. Therefore, since the door 14 can be brought close to the lower front surface 11F3 in the open position, the space in front of the housing 11 required for opening and closing the door 14 can be reduced, and the space for installing the housing 11 can be reduced and the space in front of the housing can be effectively used.

In addition, in the first technology, as shown in FIGS. 7 to 11 as an example, in the entire movement range in the opening and closing operation of the door 14, the door 14 moves while maintaining the posture of the closed position. Therefore, for example, the space in front of the housing 11 required for opening and closing the door 14 can be reduced as compared with a case in which the door 14 rotates and the posture changes during the opening operation. As a result, it is possible to reduce the space for installing the housing 11 and to effectively use the space in front of the housing 11. In addition, since the door 14 maintains the posture of the closed position in the opening and closing operation, a visual sense of unity can be maintained between the closed position and the open position.

In addition, in the first technology, as shown in FIGS. 1 to 4 as an example, the display section 13 is provided on the inclined surface 11F1. Therefore, the line of sight of the user is directed toward the inclined surface 11F1 in order to check the display content. As a result, the inclination of the inclined surface 11F1 is more strongly impressed on the user, so that the effect of providing a visual sense of unity between the inclined surface 11F1 and the movement direction of the door 14 is more conspicuous.

In addition, in the first technology, as shown in FIGS. 1 and 2 as an example, the display section 13 is a touch panel display. Therefore, since the display section 13 is used for the operation, the line of sight of the user is directed toward the display section 13 more frequently. Therefore, since the line of sight of the user is directed toward the display section 13 behind the door 14 that protrudes forward, the opening operation of the door 14 that protrudes forward is strongly impressed. As a result, a visual effect of making the working space 26 exposed to the outside by the opening operation of the door 14 feel wider is further generated. In addition, since the frequency at which the line of sight is directed toward the display section 13 is further increased, the effect of providing a visual sense of unity between the inclined surface 11F1 and the movement direction of the door 14 is more conspicuous.

In addition, in the first technology, as an example, the inclined surface 11F1 includes the display section 13 and has the low brightness region LBA having the relatively low brightness color. The color of the low brightness region LBA is a color having a lower brightness than the color of the portion other than the low brightness region LBA of the front surface 11F, the upper surface 11U, and the two side surfaces 11S of the housing 11. In addition, the color of the portion other than the low brightness region LBA is an achromatic color. Further, the color of the low brightness region LBA is black, and the color of the portion other than the low brightness region LBA is white.

By such coloring, the following effects are obtained. That is, since the black having a relatively low brightness is a representative contracting color, it appears relatively small, whereas since the white having a relatively high brightness is a representative expanding color, it appears relatively large. By such coloring of the housing 11, the contrast between the inclined surface 11F1 on which the low brightness region LBA is provided and the other portions is emphasized. Then, the line of sight of the user is likely to be concentrated on the inclined surface 11F1 including the low brightness region LBA, and on the other hand, the door 14 having a relatively high brightness descends along the inclined surface 11F1 in the opening operation, and the door 14 protrudes toward the user. The door 14 appears larger than the actual size by the coloring effect. Since the working space 26 appears by the door 14 protruding, a visual effect occurs in which the user feels that the working space 26 is wider than the actual size. As a result, favorable workability can be provided to the user psychologically.

It should be noted that, in the present example, the coloring in which the color of the low brightness region LBA is black and the color of the portion other than the low brightness region LBA is white is shown as an example, but the present disclosure is not limited thereto. For example, the color of the low brightness region LBA may be other coloring as long as the brightness is lower than the color of the portion other than the low brightness region LBA. For example, the color of the low brightness region LBA may be other than black, and may be, for example, gray. In addition, the color of the portion other than the low brightness region LBA may be an achromatic color than white, or may be an achromatic color as long as the brightness is higher than the color of the low brightness region LBA. Of course, the monochromatic coloring in which the low brightness region LBA is black and the other portions are white can give a clear and calm impression. In addition, even in a case in which the low brightness region LBA is black and the other portions are in an achromatic color, a similarly calm impression can be given.

In addition, in the first technology, as shown in FIGS. 1 and 2 as an example, in the door 14, the finger hook 21 for opening and closing the door 14 is provided on the upper side of the center in the up-down direction. Therefore, the operability is good.

### Effects of Second Technique

In the second technology of the present disclosure, as shown in FIGS. 1 to 4, the examination device 10 is an examination device that examines the specimen, in which the display section 13 and the door 14 that is openable and closable are arranged in this order from above on the front surface 11F of the housing 11. A working space 26 having a stage 27 on which the specimen is set is provided inside the door 14. The opening operation of the door 14 from the closed position toward the open position starts with an operation of moving obliquely downward and forward, and the door 14 descends toward the open position. In the opening operation, the door 14 protrudes from the display section 13 side on which the line of sight of the user is frequently directed toward the user side while descending. Since the working space 26 appears by such an opening operation, a visual effect occurs in which the working space 26 feels wider. The usability is improved by the psychological effect of the visual effect.

In addition, in the second technology, as shown in FIG. 3 as an example, in the depth direction of the housing 11, the front end 27B of the stage 27 protrudes forward of the display section 13. The opening operation of the door 14 starts with an operation of moving obliquely downward and forward. In the opening operation, the door 14 protrudes from the display section 13 on which the line of sight of the user is frequently directed toward the user side while descending. Since the stage 27 at the lower end of the working space 26 protrudes forward of the display section 13 and the opening operation of the door 14 starts with an operation of moving toward the front where the stage 27 protrudes, a visual effect of making the working space 26 feel wider is further generated.

In addition, in the second technology, as shown in FIGS. 1 and 2 as an example, the door 14 is formed over the entire width of the housing 11. Since the door 14 is formed over the entire width of the housing 11, a visual effect of making the working space 26 feel wider is further generated.

In addition, in the second technology, as shown in FIGS. 1 to 4 as an example, the examination device 10 includes the inclined surface 11F1 that is connected to the upper surface 11U of the housing 11 and is inclined rearward on the front surface 11F of the housing 11. In a case in which an angle with respect to the horizontal direction is denoted by α, the inclined surface 11F1 satisfies a condition of 90°<α<160°. Then, the display section 13 is disposed on the inclined surface 11F1. The inclined surface 11F1 on which the display section 13 is disposed in the same direction as the obliquely downward direction in which the door 14 moves at an initial stage of the opening operation is inclined. Since the door 14 moves along the inclined surface 11F1, a visual sense of unity is realized between the inclined surface 11F1 and the movement direction of the door 14, and a psychologically favorable operation feeling is provided.

It should be noted that the angle α of the inclined surface 11F1 more preferably satisfies a condition of 90°<α<145°, and still more preferably satisfies a condition of 100°<α<120°. This is because, for example, in a case in which the display section 13 is provided on the inclined surface 11F1, it is considered that the range of α is preferably at this level in consideration of the visibility of the user.

In addition, in the second technology, as shown in FIG. 4 as an example, in the open position of the door 14, the upper end surface 14U of the door 14 and the upper surface 27A of the stage 27 are in a state of being arranged in the depth direction of the housing 11. For example, the upper end surface 14U and the upper surface 27A are positioned on the same plane. Therefore, an illusion that the stage 27 is extended in the depth direction by the upper end surface 14U of the door 14 occurs, and the stage 27 can be felt to be wider. Since the working space 26 feels wider by the psychological effect of such a visual effect, the usability is improved as compared with the related art that does not have such a configuration.

In addition, in the second technology, as shown in FIGS. 3 and 4 as an example, in the closed position, the front surface portion 14F of the door 14 is positioned forward of the display section 13. Since the door 14 in which the front surface portion 14F is positioned forward of the display section 13 in the closed position protrudes forward in the opening operation, a visual effect of making the working space 26 feel wider is further generated.

In addition, in the second technology, as shown in FIGS. 1 and 3 as an example, the front surface 11F of the housing 11 has the lower front surface 11F3 adjacent to a lower side of the door 14 in the closed position. In the closed position, the front surface portion 14F and the lower front surface 11F3 are positioned on the same plane. Therefore, in the closed position, the front surface portion 14F of the door 14 and the lower front surface 11F3 of the housing 11 are flat as a whole, so that the front surface 11F of the housing 11 can be designed to be clear.

In addition, in the second technology, as shown in FIGS. 1 to 4 as an example, the display section 13 is a touch panel display. Therefore, since the display section 13 is used for the operation, the line of sight of the user is directed toward the display section 13. Therefore, since the line of sight of the user is directed toward the display section 13 behind the door 14 that protrudes forward, the opening operation of the door 14 that protrudes forward is strongly impressed. As a result, a visual effect of making the working space 26 exposed to the outside by the opening operation of the door 14 feel wider is further generated.

In addition, in the second technology, the color of the low brightness region LBA including the display section 13 and the color of the portion other than the low brightness region LBA are colored in the same manner as in the first technology as an example. Therefore, the same effects as those of the first embodiment can be obtained.

### Effects of Third Technique

In the third technology of the present disclosure, as shown in FIGS. 1 to 4, the examination device 10 has the door 14 that is openable and closable on the front surface portion 14F of the housing 11. Then, the working space 26 having the stage 27 on which the specimen is set is provided inside the door 14, and in the open position of the door 14, the upper end surface 14U of the door 14 and the upper surface 27A of the stage 27 are positioned on the same plane. Therefore, an illusion that the stage 27 is extended in the depth direction by the upper end surface 14U of the door 14 occurs, and the stage 27 can be felt to be wider. Since the working space 26 feels wider by the psychological effect of such a visual effect, the usability is improved as compared with the related art that does not have such a configuration.

In addition, in the third technology, as shown in FIG. 4 as an example, in the open position, the gap G1 between the upper end surface 14U of the door 14 and the front end 27B of the stage 27 is 10 mm or less. Therefore, since the stage 27 and the door 14 are close to each other, a visual effect of the upper end surface 14U of the door 14 being erroneously perceived as an extension of the stage 27 is more conspicuous.

In addition, in the third technology, as shown in FIG. 5 as an example, the examination device 10 includes the pair of arms 31 that support the door 14 at both ends in the width direction, and in the open position, the upper end surfaces 31U of the pair of arms 31 are in a state of being arranged at both ends of the upper surface 27A of the stage 27 in the width direction. Therefore, an illusion that the stage 27 is extended in the width direction by the upper end surfaces 31U of the pair of arms 31 occurs, and the stage 27 can be felt to be wider. Since the working space 26 feels wider by the psychological effect of such a visual effect, the usability is improved as compared with the related art that does not have such a configuration.

In addition, in the third technology, as shown in FIG. 4 as an example, in the open position, a gap G3 is generated between the lower end of the door 14 and the installation surface L on which the housing 11 is installed. Therefore, in a case in which the door 14 is opened, interference between the door 14 and the installation surface L is suppressed. As a result, a favorable operation feeling is obtained.

In addition, in the third technology, as shown in FIGS. 1 to 4 (particularly, FIG. 3) as an example, the inclined surface 11F1 that is connected to an upper surface 11U of the housing 11 and is inclined rearward on the front surface 11F of the housing 11 is provided. The inclined surface 11F1 is positioned above the door 14. Further, in the depth direction of the housing 11, the front end 27B of the stage 27 protrudes forward of the lower end of the inclined surface 11F1. An extension line at the lower end of the inclined surface 11F1 extends obliquely downward and forward, and the front end 27B of the stage 27 is positioned forward of the lower end of the inclined surface 11F1. Therefore, a visual effect of making the stage 27 feel wider occurs, and the usability is improved by the psychological effect of the visual effect.

In addition, in the third technology, as shown in FIG. 3 as an example, an opening operation of the door 14 from the closed position toward the open position starts with an operation of moving obliquely downward and forward, and the door 14 descends toward the open position. Therefore, in the opening operation, the door 14 protrudes from the display section 13 side on which the line of sight of the user is frequently directed toward the user side while descending. Since the working space 26 appears by such an opening operation, a visual effect occurs in which the working space 26 feels wider. The usability is improved by the psychological effect of the visual effect.

In addition, in the third technology, as shown in FIG. 3 as an example, the front surface 11F of the housing 11 has the lower front surface 11F3 adjacent to a lower side of the door 14 in the closed position, and in the closed position, the front surface portion 14F and the lower front surface 11F3 are positioned on the same plane. In the closed position, the front surface portion 14F of the door 14 and the lower front surface 11F3 of the housing 11 are flat as a whole, so that the design is clear.

In addition, in the third technology, the color of the low brightness region LBA including the display section 13 and the color of the portion other than the low brightness region LBA are colored in the same manner as in the first technology as an example. Therefore, the same effects as those of the first embodiment can be obtained.

The above description discloses the following technology according to First Supplementary Note.

### First Supplementary Note 1

An examination device that examines a specimen, the examination device including:
an inclined surface that is connected to an upper surface of a housing and is inclined rearward on a front surface of the housing, in which, in a case in which an angle with respect to a horizontal direction is denoted by α, a condition of 90°<α<160° is satisfied; and
a door that is openable and closable and is disposed below the inclined surface at the front surface, in which an opening operation from a closed position toward an open position starts with an operation of moving downward along the inclined surface.

### First Supplementary Note 2

The examination device according to First Supplementary Note 1,
in which, in the closed position, an upper end surface of the door is exposed, and the upper end surface is closer to horizontal than the inclined surface.

### First Supplementary Note 3

The examination device according to First Supplementary Note 2,
in which the upper end surface of the door is a horizontal surface.

### First Supplementary Note 4

The examination device according to First Supplementary Note 2 or 3,
in which, in the closed position, a plane including the upper end surface of the door is adjacent to a lower end of the inclined surface.

### First Supplementary Note 5

The examination device according to any one of First Supplementary Notes 2 to 4,
in which a front surface portion of the door that is connected to the upper end surface and extends downward is closer to vertical than the inclined surface.

### First Supplementary Note 6

The examination device according to First Supplementary Note 5,
in which the front surface portion is a vertical surface.

### First Supplementary Note 7

The examination device according to First Supplementary Note 5 or 6,
in which the front surface of the housing has a lower front surface adjacent to a lower side of the door in the closed position, and in the closed position, the front surface portion and the lower front surface are positioned on the same plane.

### First Supplementary Note 8

The examination device according to any one of First Supplementary Notes 1 to 7,
in which, in an entire movement range in an opening and closing operation of the door, a rear end of the door does not protrude forward of a front end of the door in a case in which the door is in the open position.

### First Supplementary Note 9

The examination device according to any one of First Supplementary Note 1 to 8,
in which the front surface of the housing has a lower front surface adjacent to a lower side of the door in the closed position, and
in the open position, the door covers the lower front surface on a front side of the lower front surface, and a gap between the door and the lower front surface of the housing is 10 mm or less.

### First Supplementary Note 10

The examination device according to any one of First Supplementary Notes 1 to 9,
in which, in an entire movement range in an opening and closing operation of the door, the door moves while maintaining a posture of the closed position.

### First Supplementary Note 11

The examination device according to any one of First Supplementary Notes 1 to 10,
in which a display section is provided on the inclined surface.

### First Supplementary Note 12

The examination device according to First Supplementary Note 11,
in which the display section is a touch panel display.

### First Supplementary Note 13

The examination device according to First Supplementary Note 11,
in which the inclined surface includes the display section and has a low brightness region having a relatively low brightness color, and
a color of the low brightness region is a color having a lower brightness than a color of a portion other than the low brightness region of the front surface, the upper surface, and two side surfaces of the housing.

### First Supplementary Note 14

The examination device according to First Supplementary Note 13,
in which the color of the low brightness region is black.

### First Supplementary Note 15

The examination device according to First Supplementary Note 14,
in which the color of the portion other than the low brightness region is an achromatic color.

### First Supplementary Note 16

The examination device according to any one of First Supplementary Notes 1 to 15,
in which, in the door, a finger hook for opening and closing the door is provided on an upper side of a center in an up-down direction.

### First Supplementary Note 17

The examination device according to any one of First Supplementary Notes 1 to 16,
in which a working space in which the specimen is set is provided inside the door.

### First Supplementary Note 18

The examination device according to any one of First Supplementary Notes 1 to 17,
in which the inclined surface is an inclined surface that satisfies a condition of 100°<α<120°.

In addition, the above description discloses the following technology according to Second Supplementary Note.

### Second Supplementary Note 1

An examination device that examines a specimen,
in which a display section and a door that is openable and closable are arranged in this order from above on a front surface of a housing,
a working space having a stage on which the specimen is set is provided inside the door, and
an opening operation of the door from a closed position toward an open position starts with an operation of moving obliquely downward and forward, and the door descends toward the open position.

### Second Supplementary Note 2

The examination device according to Second Supplementary Note 1,
in which, in a depth direction of the housing, a front end of the stage protrudes forward of the display section.

### Second Supplementary Note 3

The examination device according to Second Supplementary Note 2,
in which the door is formed over an entire width of the housing.

### Second Supplementary Note 4

The examination device according to Second Supplementary Note 2 or 3, including:
an inclined surface that is connected to an upper surface of the housing and is inclined rearward on the front surface of the housing, in which, in a case in which an angle with respect to a horizontal direction is denoted by α, a condition of 90°<α<160° is satisfied,
in which the display section is provided on the inclined surface.

### Second Supplementary Note 5

The examination device according to any one of Second Supplementary Note 1 to 4,
in which, in the open position of the door, an upper end surface of the door and an upper surface of the stage are in a state of being arranged in a depth direction of the housing.

### Second Supplementary Note 6

The examination device according to Second Supplementary Note 5,
in which, in the open position of the door, the upper end surface of the door and the upper surface of the stage are positioned on the same plane.

### Second Supplementary Note 7

The examination device according to any one of Second Supplementary Notes 1 to 6,
in which, in the closed position, a front surface portion of the door is positioned forward of the display section.

### Second Supplementary Note 8

The examination device according to Second Supplementary Note 7,
in which the front surface of the housing has a lower front surface adjacent to a lower side of the door in the closed position, and
in the closed position, the front surface portion and the lower front surface are positioned on the same plane.

### Second Supplementary Note 9

The examination device according to any one of Second Supplementary Notes 1 to 8,
in which the display section is a touch panel display.

### Second Supplementary Note 10

The examination device according to any one of Second Supplementary Notes 1 to 9,
in which the inclined surface includes the display section and has a low brightness region having a relatively low brightness color, and
a color of the low brightness region is a color having a lower brightness than a color of a portion other than the low brightness region of the front surface, the upper surface, and two side surfaces of the housing.

### Second Supplementary Note 11

The examination device according to Second Supplementary Note 10,
in which the color of the low brightness region is black.

### Second Supplementary Note 12

The examination device according to Second Supplementary Note 11,
in which the color of the portion other than the low brightness region is an achromatic color.

### Second Supplementary Note 13

The examination device according to Second Supplementary Note 4,
in which the inclined surface is an inclined surface that satisfies a condition of 100°<α<120°.

Further, the above description discloses the following technology according to Third Supplementary Note.

### Third Supplementary Note 1

An examination device that examines a specimen,
in which a door that is openable and closable is disposed on a front surface of a housing,
a working space having a stage on which the specimen is set is provided inside the door, and
in an open position of the door, an upper end surface of the door and an upper surface of the stage are positioned on the same plane.

### Third Supplementary Note 2

The examination device according to Third Supplementary Note 1,
in which, in the open position, a gap between the upper end surface of the door and a front end of the stage is 10 mm or less.

### Third Supplementary Note 3

The examination device according to Third Supplementary Note 1 or 2, including:
a pair of arms that support the door at both ends in a width direction,
in which, in the open position, upper end surfaces of the pair of arms are in a state of being arranged at both ends of the upper surface of the stage in the width direction.

### Third Supplementary Note 4

The examination device according to any one of Third Supplementary Notes 1 to 3,
in which, in the open position, a gap is generated between a lower end of the door and an installation surface on which the housing is installed.

### Third Supplementary Note 5

The examination device according to any one of Third Supplementary Notes 1 to 4,
in which an inclined surface that is connected to an upper surface of the housing, is inclined rearward on the front surface of the housing, and is positioned above the door is provided, and
in a depth direction of the housing, a front end of the stage protrudes forward of a lower end of the inclined surface.

### Third Supplementary Note 6

The examination device according to any one of Third Supplementary Notes 1 to 5,
in which an opening operation of the door from a closed position to an open position starts with an operation of moving obliquely downward and forward and the door descends toward the open position.

### Third Supplementary Note 7

The examination device according to Third Supplementary Note 6,
in which the front surface of the housing has a lower front surface adjacent to a lower side of the door in the closed position, and
in the closed position, a front surface portion of the door and the lower front surface are positioned on the same plane.

### Third Supplementary Note 8

The examination device according to Third Supplementary Note 5,
in which a display section is provided on the inclined surface,
the inclined surface includes the display section and has a low brightness region having a relatively low brightness color, and
a color of the low brightness region is a color having a lower brightness than a color of a portion other than the low brightness region of the front surface, the upper surface, and two side surfaces of the housing.

### Third Supplementary Note 9

The examination device according to Third Supplementary Note 8,
in which the color of the low brightness region is black.

### Third Supplementary Note 10

The examination device according to Third Supplementary Note 9,
in which the color of the portion other than the low brightness region is an achromatic color.

In addition, in the present embodiment, the “vertical” indicates vertical in a meaning including an error that is generally allowed in the technical field to which the technology of the present disclosure belongs, and an error to such an extent not contrary to the spirit of the technology of the present disclosure, in addition to completely vertical. In addition, similarly, the “horizontal” indicates horizontal in a meaning including an error that is generally allowed in the technical field to which the technology of the present disclosure belongs, and an error to such an extent not contrary to the spirit of the technology of the present disclosure, in addition to completely horizontal.

The technology of the present disclosure can also be combined with various embodiments and/or various modification examples described above, as appropriate. In addition, it goes without saying that the present disclosure is not limited to each of the embodiments described above, various configurations can be adopted as long as the configuration does not deviate from the gist.

The above-described contents and the above-shown contents are the detailed description of the parts according to the technology of the present disclosure, and are merely an example of the technology of the present disclosure. For example, the descriptions regarding the configurations, the functions, the actions, and the effects are descriptions regarding an example of the configurations, the functions, the actions, and the effects of the part according to the present disclosed technology. Accordingly, it goes without saying that unnecessary parts may be deleted, new elements may be added, or replacements may be made with respect to the above-described contents and the above-shown contents within a range that does not deviate from the gist of the technology of the present disclosure. In addition, in the above-described contents and the above-shown contents, the detailed description of, for example, common technical knowledge that does not need to be particularly described to enable the implementation of the technology of the present disclosure is omitted in order to avoid confusion and facilitate the understanding of portions related to the technology of the present disclosure.

In the present specification, “A and/or B” has the same meaning as “at least one of A or B”. That is, the “A and/or B” means that it may be only A, only B, or a combination of A and B. In addition, in the present specification, also in a case where three or more matters are expressed in association by “and/or”, the same concept as “A and/or B” is applied.

All of the documents, the patent applications, and the technical standards described in the present specification are incorporated herein by reference to the same extent as in a case where each of the documents, patent applications, and technical standards is specifically and individually described by being incorporated by reference.

Explanation of References

What is claimed is:

1. An examination device that examines a specimen, the examination device comprising:
an inclined surface that is connected to an upper surface of a housing and is inclined rearward on a front surface of the housing, in which, in a case in which an angle with respect to a horizontal direction is denoted by α, a condition of 90°<α<160°is satisfied; and
a door that is openable and closable and is disposed below the inclined surface at the front surface, in which an opening operation from a closed position toward an open position starts with an operation of moving downward along the inclined surface.

2. The examination device according to claim 1,
wherein, in the closed position, an upper end surface of the door is exposed, and the upper end surface is closer to horizontal than the inclined surface.

3. The examination device according to claim 2,
wherein the upper end surface of the door is a horizontal surface.

4. The examination device according to claim 2,
wherein, in the closed position, a plane including the upper end surface of the door is adjacent to a lower end of the inclined surface.

5. The examination device according to claim 2,
wherein a front surface portion of the door that is connected to the upper end surface and extends downward is closer to vertical than the inclined surface.

6. The examination device according to claim 5,
wherein the front surface portion is a vertical surface.

7. The examination device according to claim 5,
wherein the front surface of the housing has a lower front surface adjacent to a lower side of the door in the closed position, and
in the closed position, the front surface portion and the lower front surface are positioned on the same plane.

8. The examination device according to claim 1,
wherein, in an entire movement range in an opening and closing operation of the door, a rear end of the door does not protrude forward of a front end of the door in a case in which the door is in the open position.

9. The examination device according to claim 1,
wherein the front surface of the housing has a lower front surface adjacent to a lower side of the door in the closed position, and
in the open position, the door covers the lower front surface on a front side of the lower front surface, and a gap between the door and the lower front surface of the housing is 10 mm or less.

10. The examination device according to claim 1,
wherein, in an entire movement range in an opening and closing operation of the door, the door moves while maintaining a posture of the closed position.

11. The examination device according to claim 1,
wherein a display section is provided on the inclined surface.

12. The examination device according to claim 11,
wherein the display section is a touch panel display.

13. The examination device according to claim 11,
wherein the inclined surface includes the display section and has a low brightness region having a relatively low brightness color, and
a color of the low brightness region is a color having a lower brightness than a color of a portion other than the low brightness region of the front surface, the upper surface, and two side surfaces of the housing.

14. The examination device according to claim 13,
wherein the color of the low brightness region is black.

15. The examination device according to claim 14,
wherein the color of the portion other than the low brightness region is an achromatic color.

16. The examination device according to claim 1,
wherein, in the door, a finger hook for opening and closing the door is provided on an upper side of a center in an up-down direction.

17. The examination device according to claim 1,
wherein a working space in which the specimen is set is provided inside the door.

18. The examination device according to claim 1,
wherein the inclined surface is an inclined surface that satisfies a condition of 100°<α<120°.
